(19) United States
(12) Patent Application Publication
(10) Pub. No.: US 2024/0316343 A1
(43) Pub. Date: Sep. 26, 2024

(54) PROCESS AND SYSTEM FOR ARTIFICIAL ACTIVATION AND MONITORING OF NEUROMUSCULAR TISSUE BASED ON ARTIFICIAL INTELLIGENCE

(71) Applicants: Ângelo Eustáquio ZANDONA FREITAS, Belo Horizonte (BR); Bruno Silveira AVELAR, Belo Horizonte (BR); Daniel Alexandre SANTOS, Nova Lima (BR); Diógenes De Oliveira ALCÂNTARA, Belo Horizonte (BR); Humberto Jonas Fátima ABRÃO, Nova Lima (BR); Paulo Eugênio Oliveira DE SOUZA E SILVA, Brasília (BR); Pedro Villaça DE ALMEIDA, Nova Lima (BR)

(72) Inventors: Ângelo Eustáquio ZANDONA FREITAS, Belo Horizonte (BR); Bruno Silveira AVELAR, Belo Horizonte (BR); Daniel Alexandre SANTOS, Nova Lima (BR); Diógenes De Oliveira ALCÂNTARA, Belo Horizonte (BR); Humberto Jonas Fátima ABRÃO, Nova Lima (BR); Paulo Eugênio Oliveira DE SOUZA E SILVA, Brasília (BR); Pedro Villaça DE ALMEIDA, Nova Lima (BR)

(21) Appl. No.: 17/791,889

(22) PCT Filed: Jan. 13, 2020
(86) PCT No.: PCT/BR2020/050005

§ 371 Date: Jul. 9, 2022

(51) Int. Cl. A61N 1/36; A61N 1/375; G16H 40/00; G16H 50/20

(52) U.S. Cl. CPC A61N 1/36031; A61N 1/36034; A61N 1/375; G16H 40/00; G16H 50/20

(57) ABSTRACT

The invention is characterized by a set of innovations in the technique called Artificial Neuromuscular Activation and Monitoring System—SAMNA, based on artificial intelligence. The invention automatically enables accurate diagnosis and effective personalized treatment for each type of muscle group, in different patients, allows continuous monitoring of the neuromuscular condition, treating and preventing vascular, osteotendinous and neuromuscular disorders with artificial and automated neuromuscular activation using cloud data processing and machine learning, allows the diagnosis/treatment of neuromuscular disorders on a continuous basis, locally or remotely. The hardware, firmware and software resources allow the acquisition of neurophysiological parameters and application of therapeutic parameters automatically, eliminating the use of sensors, making use of the electrotherapy electrodes themselves and performing their actions independently, personalized and coordinated, continuously controlling the contraction level.

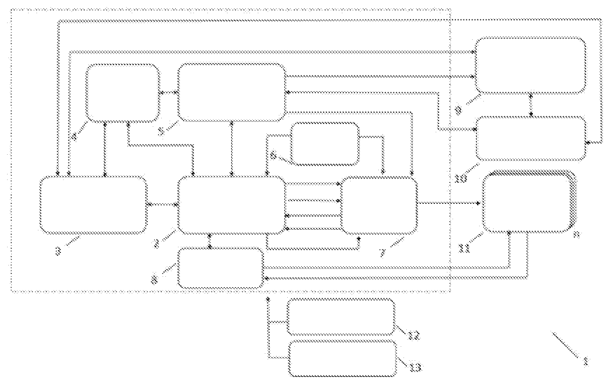

### FIELD OF THE INVENTION

The present invention belongs predominantly to the health area, with a focus on physiotherapy applications aimed at activating neuromuscular tissues. In a complementary way, the technology developed is also located in the field of Information and Communication Technologies (ICTs), as it comprises knowledge related to devices that employ artificial intelligence and the internet of things (IoT—internet of things). More particularly, the invention addresses matter pertaining to automatic, non-invasive equipment, aimed at continued neuromuscular electrical activation.

The developed technology consists of a system equipped with an IoT resource, with horizontal monitoring of the neuromuscular condition, processing of data in the cloud, from databases using knowledge of big data, also employing learning techniques machine, also known as machine learn and technique are to prevent, treat and diagnose disorders of the neuromuscular system by means of neurophysiological tests and artificial neuromuscular activation. All of these actions are carried out automatically, continuously, locally or remotely. For this, the system has hardware, firmware and software resources that allow the control of therapeutic and diagnostic parameters, ensuring the appropriate adjustments for each muscle group.

The invention, through the listed resources, makes it feasible to apply a more effective, customized and personalized dose for each muscle group of each patient. The invention is also capable of executing different therapeutic protocols that are pre-programmed in the system and new protocols can be loaded remotely from a server that can be in a cloud structure and that is an integral part of the system. These same therapeutic protocols can also be updated automatically using a local artificial intelligence algorithm, executed by a specific and dedicated processor. The parameterization of the local neural network can also be altered by the action of a top layer of artificial intelligence executed in the cloud. At this level, the global dynamics and

[symbol]

1. SYSTEM FOR ENABLING AND MONITORING BASED ON ARTIFICIAL neuromuscular ARTIFICIAL INTELLIGENCE comprising one structural cart with wheels and support structure (45) case (34) provided with adapter hardware (31), power source (32), converter DC DC (33), an articulated arm (41) takes the case of connection of electrodes (47) with the connectors of cables electrical stimulation (35), a man-machine interface (5) to display touch screen (39) and a display controller board (43) allocated n the cabinet (34), the monitor (40) and provided with articulated arm (41), for moving case of connection of electrodes (47), on-off button (36) and connection ethernet (38) located in the parameter panel (42), emergency button (37) and knob type button (46) for navigation.

2. SYSTEM, according to claim 1, characterized by comprising mechanisms of continuous and simultaneous stimulation of osteotendinous and vascular tissues, said mechanisms promoting electrical activation of peripheral or central nervous tissue, and muscle tissue from different dermatomes in a manner automatic.

3. SYSTEM according to claim 1, characterized in that is provided with engine control d current density power applied (in mA/centímetro²) maintaining said density equal to or less than 3.5 m A/cm².

4. SYSTEM according to claim 1, characterized by an artificial intelligence algorithm built, with an automatic calibration and therapeutic protocols performed by means of said allocated server algorithm in cloud structure.

5. PROCESS, according to claim 1, characterized by comprising the activation of the central processing unit (2) by means of commands sent to the impedance processor (26) and delivered by the communication device (25) to the myography system by electrical impedance (8) for reading the myographic activity using pairs of electrodes (11) at the [symbol]
